(19) United States
(12) Patent Application Publication
(10) Pub. No.: US 2009/0298773 A1
(43) Pub. Date: Dec. 3, 2009

(54) METHODS OF PRODUCING FUNCTIONAL MISO

(75) Inventors: Hirokuni KITAJIMA, Nagano-shi (JP); Takeshi NAKAZAWA, Nagano-shi (JP); Morio OKADA, Nagano-shi (JP); Kaori INOUE, Sagamihara-shi (JP); Naoyuki YAMAMOTO, Sagamihara-shi (JP); Takanobu GOTO, Tokyo (JP)

Correspondence Address: BIRCH STEWART KOLASCH & BIRCH, PO BOX 747, FALLS CHURCH, VA, 22040-0747, US

(73) Assignees: Marukome Co., Ltd., Nagano-shi (JP); Calpis Co., Ltd., Tokyo (JP)

(21) Appl. No.: 12/466,992

(22) Filed: May 15, 2009

(30) Foreign Application Priority Data
Nov. 17, 2006 (JP) 2006-311264
Nov. 16, 2007 (JP) PCT/JP2007/200722

(51) Int. Cl. A61K 38/16; A23L 1/30; A23L 2/66; A23L 1/20; A23L 1/305

(52) U.S. Cl. 514/12; 426/46; 426/589; 426/634; 426/648

(57) ABSTRACT

According to the present invention a miso with good flavor and a processed food containing the miso are provided, wherein the miso and the food are expected to have an repressing effect on an increase in blood pressure or hypotensive effect by continually taking the miso or food for routinely dietary habit. The present invention also provides a method of producing a miso, wherein peptides with ACE inhibitory activity are effectively produced in the miso without lowering the quality in flavor of miso. In an embodiment of the present invention, animal milk is added at a content of 10% during a mixing step, or a fermentation and ripening step.

### FIELD OF THE INVENTION

The present invention relates to a novel miso, particularly, a miso having a hypotensive effect and a method of producing thereof.

The present invention also relates to a processed food containing the miso of the present invention and a method of producing the food.

### BACKGROUND OF THE INVENTION

To date various peptides with a hypotensive effect have been reported and isolated from enzymolytic products of proteins for foods and microbiologically fermented foods. Among such peptides, peptides having proline (Pro) residue at the carboxyl terminal or in the internal sequences are demonstrated to encompass many peptides which modulate blood pressure in vivo, that is, those having an angiotensin converting enzyme (ACE) inhibitory effect and contribute to exertion of the in vivo hypotensive effect. Regarding such peptides having ACE inhibitory activity, tripeptides such as VPP (valine-proline-proline), IPP (isoleucine-proline-proline), LPP (leucine-proline-proline), LKP (leucine-lysine-proline) and dipeptides such as VY (valine-tyrosine) are known, but are not limited to them. The features shared by the known ACE inhibitory peptides include, a molecular weight of 500 or less, bonded 2 to 5 amino acid residues and proline at the second and/or third amino acid residue from the amino-terminal.

Many of these peptides with ACE inhibitory activity having hypotensive effect are known to have a strong blood pressure controlling effect on spontaneously hypertensive rats (Yamamoto et al. (2003) Current Pharmacol. Des. 9: 1345-1355). The different peptides with ACE inhibitory activity have been produced by enzymatically hydrolyzing proteinous materials for foods. The proteinous materials generally include, milk proteins, soybean proteins, egg proteins, proteins of wheat, fish or butcher's meat.

Representative microorganisms for use in foods include lactic acid bacteria used in yoghourt, yeast used in bread or brewing and Bacillus natto used in “natto” (fermented soybeans), which are all well known. It has been also reported that a fermented product, such as fermented milk by lactic acid bacteria, had ACE inhibitory activity (JP-06-40944).

Recently, a method of enriching peptides with ACE inhibitory activity in a miso was reported, where powdered skimmed milk was admixed and the miso is ripened during the production of miso (WO 2005/074712, Muguruma et al, Zoukyo, Vol. 100 (2005) p. 216-223). Muguruma et al. (2005) reported that for the miso produced by adding 16% of skimmed milk angiotensin converting enzyme inhibitory activity was enhanced about 4-fold as compared with a normal miso.

Dietary habit of eating miso have a long history and quality loss such as change and/or deterioration in flavor due to the alteration in the method of production would therefore be a great problem for routine eating. For example, in the sensory test described in Muguruma et al. (2005), the results demonstrated that the proportion of the panelists who had uncomfortable feeling for fermented miso with additive skimmed milk was high, which means there is a need for a technique in cooking. Additionally, common salt is usually added during the production of miso, it is necessary to reduce the amount of the additive common salt as low as possible or increase productivity of the peptides with ACE inhibitory activity as high as possible during the production of miso. Thus, development of techniques for repressing changes in flavor and enhancing the productivity of the peptides with ACE inhibitory activity have been essentially desired.

### SUMMARY OF THE INVENTION

The present invention therefore provides a miso with good flavor and a processed food containing the miso, wherein the miso and the food are expected to have an repressing effect on an increase in blood pressure or hypotensive effect by continually taking the miso or food for routinely dietary habit.

An object of the present invention is also to provide a method of producing a miso, wherein peptides with ACE inhibitory activity are effectively produced in the miso without lowering the quality in flavor of miso.

The present inventors observed that peptides with ACE inhibitory effect could be effectively produced in a miso by adding a proteinous materials, particularly animal milk, at a content of 10% during a mixing step, or a fermentation and ripening step in the production of miso without lowering the quality in flavor of miso, investigated essential conditions required for the fermentation and ripening of miso based on the observation and developed the miso of the present invention and the method of producing thereof.

Without wishing to be bound by any theory, the reasons for the aforementioned observation may be achieved by use of casein instead of the powdered skimmed milk to repress production of byproducts with specific abnormal odor derived from galactose or other components in whey and to effectively hydrolyze casein which is suitable as a material for peptides with ACE inhibitory activity.

Such produced miso of the present invention, the method of producing the miso and a processed food containing the miso and the method of producing them are described hereinafter.

The miso of the present invention is characterized in that it contains a brew into which animal casein has been added at a content of 10% by weight or more during a mixing step or a fermentation and ripening step.

Preferably 90% or more of the total peptides in the brew are peptides having a molecular weight of 500 or less.

Preferably proline content in amino acids composing of the total peptides in the brew is 15% or more.

More preferably, the peptides are those having Pro at the second and/or the third amino acid residues at a content of 15% or more in the total peptides contained in the brew.

More preferably, the brew has the angiotensin converting enzyme inhibitory activity of 550 U/g or more.

The method of producing the miso of the present invention is characterized in that animal casein is added at a content of 10% by weight or more during the mixing step or the fermentation and ripening step.

The method of producing the miso-containing food of the present invention is characterized in that seasonings and/or foods are added into the miso of the present invention such that the food has the angiotensin converting enzyme inhibitory activity of 7,800 Upper meal or more.

The method preferably further comprises powderization of the miso.

More preferably the powderization is conducted by freeze-drying.

In the miso containing-food of the present invention, the miso is preferably powdered.

According to the present invention, a miso with a good flavor is provided, wherein the ACE inhibitory activity of the miso is increased 7-fold or more as compared with a normal miso.

According to the present invention, aforementioned miso can be produced by adding animal casein into raw materials of miso during the mixing step or the fermentation and ripening step. A miso-containing processed food with improved utility and storage stability can be produced by adding seasonings and/or foods, or by further processing them by, for example, freeze-drying.

Additionally, according to the present invention a miso with ACE inhibitory activity of 550 U/g or more may be provided. Furthermore, various processed misos with 7,800 Upper meal as an effective amount is also provided.

Various functional foods and foods for medical use as well as methods of producing them are also provided, because an effect of repressing blood pressure in hypertensives is expected for the miso of the present invention via ingestion of the miso as a routine dietary component, and a hypotensive effect on those having high value of blood pressure is also expected.

### BRIEF DESCRIPTION OF THE DRAWINGS

FIG. 1 shows the comparison of various misos (casein-miso, normal miso and skimmed milk-miso) for ACE inhibitory activities (Example 1).

FIG. 2 shows the relationship between concentration of additive casein and ACE inhibitory activity of the casein-miso (Example 1).

FIG. 3 shows the analysis of the molecular sizes of the casein-miso peptides by LC/MS (Example 2).

FIG. 4 shows the comparison of hypotensive activities of various miso on spontaneously hypertensive rat (SHR). The denotations in the figure (“*” and “#”) represent the significant differences for each casein miso in t-test (*: P<0.05, #: P<0.1).

### DESCRIPTION OF THE PREFERRED EMBODIMENTS

The miso of the present invention obtained by adding casein include, for example rice-miso of which raw materials are rice-koji, steamed soybean and common salt, “mugi” (barley)-miso of which raw materials are barley, soybean-miso of which materials are soybean-koji and common salts, a combination of the rice-miso, barley-miso and soybean-miso, or a blended miso produced by combining different koji, but any miso may be encompassed.

The present miso may be produced according to any methods used in the art for producing any of the aforementioned misos. Specifically, the method of producing the miso of the present invention comprises a koji-preparation step where koji-mold is added to steamed rice, cereals such as barley or soybean and cultured to produce rice-koji, barley-koji or soybean-koji; a mixing step where steamed rice, cereals such as barley, or soybean, either of the kojis obtained by the preceding step, common salt, water, other optional materials are mixed at a desired ratio to obtain a koji-mixed material; and a fermentation and ripening step where the koji-mixed material is fermented and ripened to obtain a brew, wherein the method is characterized in that casein and/or a salt of casein is added during the mixing step or the fermentation and ripening step.

The casein or salts thereof to be added may be any of those derived from foods, with casein or salts thereof being preferably casein from animal milk. The animal milk includes, for example, milk of a farm animal such as cow, goat, sheep or horse, with milk of cow being preferable. The salts of casein include sodium casein, calcium casein and the line, with sodium casein being particularly preferable. Casein is a phosphate protein, which is classified to αₛ-casein (αₛ₁-casein, αₛ₂-casein), β-casein, γ-casein, κ-casein or the line. As use herein, “casein” refers to any phosphate proteins classified as casein including αₛ-casein (αₛ₁-casein, αₛ₂-casein), β-casein, γ-casein and κ-casein as well as the salts thereof.

In the context of the present invention, since the presence of casein in the raw materials for miso at any time of the period between the mixing step to the fermentation and ripening step is sufficient, the time point for adding casein is not particularly limited, but it is operationally preferable to add casein during the mixing step.

According to the present invention, 10% by weigh or more of casein is added to either type of raw materials for miso as described above and the mixture is fermented and ripened. The mount of additive casein is preferably 10% by weight or more, more preferably 10-18% by weight and most preferably 12-16% by weight.

The materials except for casein, which are used in the method of the present invention, may be any materials that are used in this technical field for producing any of miso. For example rice and “mugi” (barley) are representative cereals used as materials for miso, but other cereals including corn, buckwheat, Hungarian grass or Japanese millet may be also used. As “mugi” (a collective term for edible plants belonging to family Poaceae except for rice plant, including wheat, barley, naked barely, rye, oat and the like) any of edible “mugi” such as barely, naked barley, adlay (Coxi lacryma-jobi var. ma-yuen), rye and wheat may be used. Although soybean is representative as a bean used for materials of miso, any of edible beans such as mung bean, pea, black soybean, azuki bean or broad bean may be used. These cereals and beans may be used independently of cultivars or localities.

The koji-mold which is added and cultured to obtain various koji may be any koji-mold used in this technical filed for producing miso, and Aspergillus oryzae, Aspergillus sojae, Aspergillus tamari et al. are preferable. The koji-mold may be added as a form of seed koji into steamed cereals and/or beans. The temperature for adding and culturing the koji-mold depends on the optimal growing temperature of used koji-mold, and for example, the temperature is 20-45° C., preferably 25-40° C.

The salt used as a material for miso may be any edible salts including, for example, common salt (NaCl 99% or more), high grade salt (NaCl 99.5% or more), rock salt, coarse salt (NaCl 95% or more, water content of about 1.4%) and white salt (NaCl 95% or more, smaller average particle size and less water content than coarse salt).

The optional materials may include salt-resistant yeasts, salt-resistant lactic acid bacteria, which are further added and admixed during the mixing step to obtain a koji-mixed material. The salt-resistant yeast include, but are not limited to, Zygosaccharomyces rouxii, Candida versatilis, Candida etchellsii and the like, and the salt-resistant lactic acid bacteria include, but not limited to, Tetragenococcus halophilus, Pediococcus acidilactici and the like. The optional materials and the amount thereof to be added may be determined according to technical common knowledge of those skilled in the art depending the types of desired miso.

Materials except for casein (cereals, beans, salts and optional materials) may be formulated at a desired ratio depending on the types of miso to be produced. For example, the content of koji in the composition is normally 15-50% by weight (5 to 20 parts of koji to 10 parts soybean), preferably 20-30% by weight (10 parts of koji to 10 parts soybean). The content of salt in the composition is suitably 5% by weight or more, preferably 9-12% by weight. The desired final water content of the koji-mixed material may be adjusted to the range between 35% by weight and 55% by weight, taking water content of all materials used into account.

To the koji-mixed material obtained by the mixing step casein or a salt thereof is added during the mixing step or the fermentation and ripening step, the mixture is then fermented and ripened to produce a brew, that is, a miso. According to the method of the present invention, the fermentation and ripening step is conducted at 10-60° C., preferably 25-35° C. or 45-55° C.

In mid-course of the fermentation and ripening step, stirring may be conducted as appropriate. The period of the fermentation and ripening step depends on the ripening temperature and is, for example, about one month to one year at 25-35° C. and about one day to 7 days at 45-55° C.

Concerning peptides contained in a miso obtained by the method of producing miso according to the present invention (such peptide(s) are hereinafter also called as “miso peptide(s)”), peptides having the molecular weight of 500 or less should preferably occupy 50% or more of the whole peptides, and more preferably 90% or more. Furthermore, the average molecular weight of the peptides having the molecular weight of 500 or less in the whole peptides of the present invention is preferably 200 to 400.

The proline percentage in the amino acids constituting the whole peptides of a miso yielded by the production method of the present invention is preferably 5% or higher, and more preferably 15% or higher.

The miso obtained by the production method according to the present invention includes peptides, in which 10% or more, further preferably 15% or more, of the second and/or third position amino acids from the amino terminus are occupied by proline among all other amino acid residues.

The miso yielded by the production method according to the present invention has high ACE inhibitory activity. The miso of the present invention has preferably the ACE inhibitory activity of 550 U/g or higher, and more preferably the ACE inhibitory activity of 800 U/g or higher.

The miso obtained by the production method according to the present invention having high ACE inhibitory activity presumably contains a peptide with ACE inhibitory activity at a high concentration, as determined by the molecular weight and amino acids of the miso peptide.

The miso obtained by the production method according to the present invention can be mixed with various seasonings, such as sugar, salt, soy sauce, sake and a miso produced by other production method, as well as other various food materials to produce a processed food. Examples of such processed foods include, but not limited to, starting with an instant miso soup, an instant noodle or confectionery. For producing a processed food, the miso according to the present invention may be processed to a powder form by freeze-drying, etc., singly or in combination with a flavoring or a food material.

The miso according to the present invention may be processed to and/or utilized in many other food forms.

Therefore, according to the present invention a miso or a processed food containing the same with an expected in vivo hypotensive activity may be provided. Where the miso is processed expecting the activity in a human, it is preferable to process the miso to a food form such that ACE inhibitory activity of, for example, 7,800 Upper meal, can be ingested.

Alternatively, since the miso according to the present invention can be processed to and/or utilized in many food forms, a targeted daily ingestion dose can be ingested by a single meal or by a plurality of ingestions of the divided daily dose.

Concerning the amount for ingestion of a food having ACE inhibitory activity required to exert hypotensive activity in a human (effective dose), there have appeared several reports. For example, there is a report that by consuming for 8 weeks a test food prepared by processing and formulating a milk fermented by lactic acid bacteria, Lactobacillus helveticus CM4 strain, the blood pressure of a moderately hypertensive person decresed significantly (Hirata, et al., J. New Rem. & Clin. Vol. 51, 60-69). From the estimated amount of proteins in the test food, the amount of the CM4 fermented milk contained in 120 g of the test food is estimated at about 60 mL. It is further reported that the ACE inhibitory activity of the CM4 fermented milk is 130 U/mL (Yamamoto, et al., JP 09-277949 A). Calculation of these existing clinical test results indicates that it is desirable for exerting hypotensive activity in a hypertensive to take daily the CM4 fermented milk having the ACE inhibitory activity of 130 U/mL×60 mL=7,800 U or more.

Diverting the above to the miso of the present invention, hypotensive activity can be expected, when an amount of the miso containing 7,800 U of ACE inhibitory activity is ingested daily. Assuming that the ACE inhibitory activity of a miso of the present invention is 550 U/g, hypotensive activity may be expected by taking daily target ingestion amount which is calculated to be 14.2 g. However, it is also reported that continuous intake of a peptide hypotensive activity can enhance the efficacy (Mizuno, et al. Br. J. Nutr., 94, 84-91, 2005), and the activity on blood pressure may be expected at a lower dose, therefore the above value is not definite.

As shown by the result of the following Example 6, the miso produced according to the production method of the present invention was confirmed to have hypotensive activity via in vivo tests. There is another clinical test result reporting that hypotensive activity was confirmed by administering 2 mL of a milk fermented by lactic acid bacteria to 20-week-old male spontaneously hypertensive rats (SHR) (estimated body weight 330 g) (JP 06-40944 A). Further, it is reported that hypotensive activity was also confirmed by administering 95 mL of a milk fermented by the same lactic acid bacteria to a human (Hata, et al., Am. J. Clin. Nutr., 64, 767-771, 1996). According to a calculation based on the findings of these clinical test results and the result of the Example 6 of the present invention, when a human ingests the casein miso according to the present invention, the hypotensive activity can be expected by daily ingestion of 28.5 g per day (0.6 g is multiplied by a conversion coefficient for from SHR to a human, i.e. 95 mL/2 mL=47.5). However the administration amount of the casein miso used in the Example 6 does not determine the minimum effective dose with respect to the hypotensive activity in a SHR, and in view of the above-mentioned ACE inhibitory activity, the ingestion of the casein miso of the present invention at an amount less than the calculated effective dose mayso be expected to be effective in a human.

The present invention will now be described in more detail by way of Examples and comparative examples, provided that the scope of the present invention is not limited thereto.

### EXAMPLES

### Example 1

### Evaluation of ACE Inhibitory Activities of Various Misos

### [Methods of Producing Various Misos]

Rice was immersed in water overnight, then drained for about one hour and steamed for 30 minutes without pressurizing. After cooling the steamed rice, seed koji-mold (Aspergillus oryzae) for miso was added to the steamed rice at a ratio of 0.01%, and the step of preparing koji was started at 30° C., and turning and mixing (upsetting) was conducted after 18 hours and 36 hours such that the temperature of the koji does not exceed 40° C. to obtain final koji preparation to obtain a final koji after about 40 hours. Soybean was immersed in water overnight, drained and steamed for 15 minutes at 1.2 kg/cm². Using the resulting rice-koji, steamed soy bean, common salt (NaCl 99% or more), yeast (Zygosaccharomyces rouxii) and sodium casein or powdered skimmed milk, mixing step was conducted using 14 parts of koji, targeted water content of 45.2% at the mixing step, targeted common salt content of 10.8% and sodium casein or powdered skimmed milk content of 16%. For a normal miso without adding sodium casein or powdered skimmed milk, other materials were used to compensate the composition ratio of sodium casein or powdered skimmed milk.

After the materials for various miso were mixed according to the ration shown in Table 1, table 1 (I) and Table 1 (II), the fermentation and ripening were conducted at 30° C. for 60 days to produce the normal miso, skimmed milk miso of Muguruma et al. (2005) where part of raw materials of miso was replaced with powdered skimmed milk, and the casein miso into which casein was added according to the present invention.

TABLE 1
Composition of rice-miso formulation for mixing step

| | water content | |
|---|---|---|
| raw material | in raw material (%) | content ratio (%) |
| rice-koji | 21.9 | 20.4 |
| steamed soybean | 57.5 | 28.5 |
| common salt | 0 | 10.8 |
| water | 100 | 24.2 |
| yeast | 100 | 0.1 |
| casein or | 5 | 16.0 |
| powdered skimmed milk | | |
| total | 45.2 | 100 |

TABLE 1(I)
Composition of the formulation in the production of various misos

| | normal | skimmed | casein |
|---|---|---|---|
| raw material (%) | miso | milk miso | miso |
| rice-koji | 27.9 | 20.4 | 20.4 |
| steamed soybean | 53.6 | 28.5 | 28.5 |
| common salt | 12.0 | 10.8 | 10.8 |
| water | 6.4 | 24.2 | 24.2 |
| yeast | 0.1 | 0.1 | 0.1 |
| casein or | 0.0 | 16.0 | 16.0 |
| powdered skimmed milk | | | |

TABLE 1 (II)
Casein concentration in the casein miso

| raw material (%) | casein content (%) in casein miso | | | | |
|---|---|---|---|---|---|
| level of casein addition | 0% | 7.2% | 12% | 18% | 24% |
| rice-koji | 27.9 | 27.9 | 27.9 | 27.9 | 27.9 |
| steamed soybean | 53.6 | 37.5 | 26.8 | 13.4 | 0.0 |
| common salt | 12.0 | 12.0 | 12.0 | 12.0 | 12.0 |
| water | 6.4 | 15.3 | 21.2 | 28.6 | 36.0 |
| yeast | 0.1 | 0.1 | 0.1 | 0.1 | 0.1 |
| casein | 0.0 | 7.2 | 12.0 | 18.0 | 24.0 |

### [Methods of Isolating Miso-Peptides (Peptides in Miso)]

Miso was dissolved in distilled water at 400 mg/ml, sufficiently suspended by sonication, centrifuged for 10 minutes at 3000 rpm, and then the supernatant was recovered. Ten percent trichroloacetate solution (10% TCA solution) was added to the supernatant at an equivalent amount, which was left for 10 minutes at a room temperature to denature protein ingredients by acid and precipitate them and then centrifuged for 10 minutes at 15,000 rpm to recover peptides in the supernatant.

The supernatant was diluted 10-fold with distilled water and 30 ml of the dilution was applied to Sep-Pak tC-18 column which had been washed with 100% methanol and distilled water and activated. The column was washed with 7 ml of distilled water, the absorbed fraction was eluted with 2.5 ml of 30% methanol and concentrated by centrifugation under reduced pressure. The resulting concentrate was dissolved in 1.5 ml of distilled water to obtain various miso-peptides.

### [Determination of ACE Inhibitory Activity]

The determination was conducted pursuant to the method of Cushman and Cheung (D. W. Cushman and H. S. Cheung, Biochem. Pharmacol., 20 1637 (1971)).

Briefly, such prepared various miso-peptides were diluted 8-, 16- and 32-fold or 32-, 64- and 128-fold with 0.1 M borate buffer (containing 0.3 M NaCl, pH8.3), and 0.08 ml of each of them was placed into a tube. To each tube 0.2 ml of Hippuryl-His-Leu acetate salt (Sigma) prepared to be 5 mM in 0.1 M borate buffer (containing 0.3 M NaCl, pH8.3) was added as a substrate, then 0.02 ml of 0.1 U/ml of ACE solution (Angiotensin Converting Enzyme 0.1 U, Sigma) prepared in 0.1 M borate buffer (containing 0.3 M NaCl, pH8.3) was added, which was allowed to react at 37° C. for 50 minutes. The reaction was stopped by adding 0.25 ml of 1 N HCl, 0.7 ml of ethyl acetate was then added and agitated for 20 seconds. Then 1.4 ml of the ethyl acetate phase was recovered, which was heated at 120° C. for 40 minutes to remove the solvent. After removing the solvent, 1 ml of distilled water was added and absorbance at 228 nm of the extracted hippurylic acid was measured. The ACE inhibitory activities were calculated from these values according to the following formula.

From the results, final concentration of various miso-peptides solution in the reaction exhibiting 50% inhibition (=IC₅₀) was calculated as a concentration of miso in the solution. Additionally, ACE inhibitory activity in 1 g of miso (U/g) was calculated by assuming the ACE inhibition titer of miso-peptides exhibiting IC₅₀ value as one unit (1 U).

Inhibition rate (%)=[(A−B)/A]×100(%)

wherein,
A: Absorbance at 228 nm without sample (miso-peptides)
B: Absorbance at 228 nm with sample (miso-peptides)

### [Comparison of ACE Inhibitory Activities of Miso-Peptides]

As shown in Table 2, the ACE inhibitory activities were compared for the skimmed milk-miso according to the method described in Reference 2 (added powdered skimmed milk content of 16%, NaCl concentration of 10.8%, water content of 45.2%, ripening period of 60 days), the casein-miso according to the present invention produced under the condition of additive casein content of 16%, NaCl concentration of 10.8%, water content of 45.2% and ripening period of 60 days and a normal miso (NaCl concentration of 12.0%, water content of 43.0%, ripening period of 60 days) as a control. The results were shown in the drawings.

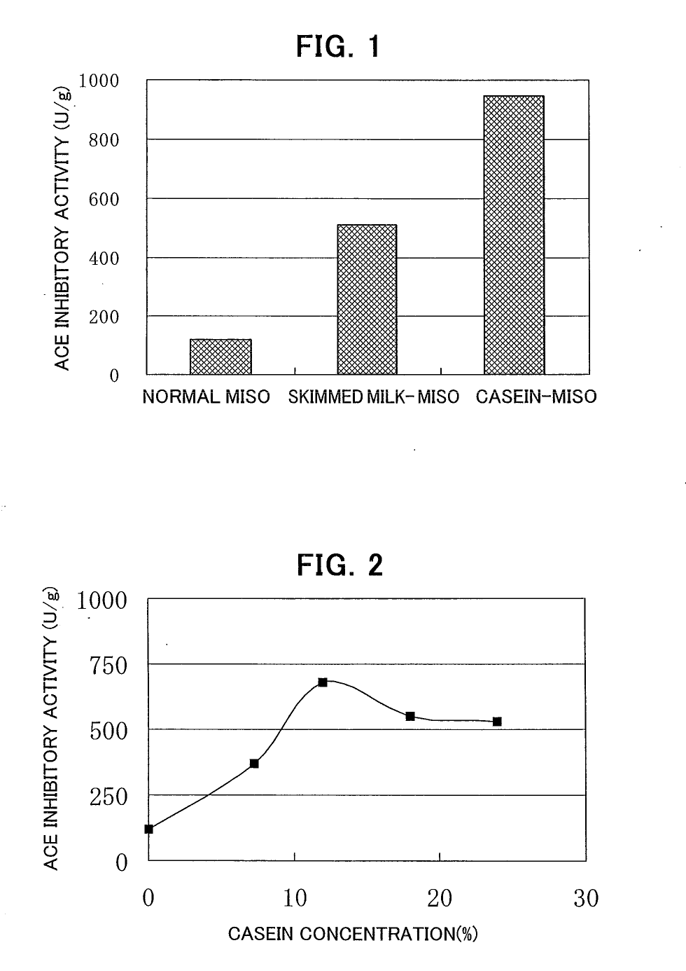

As shown in FIG. 1 and Table 3, the ACE activity of normal miso was 122 U/g, while the ACE activity the miso containing powdered skimmed milk (skimmed milk-miso) produced according to the method of Reference 2 was 511 U/g, and the ACE inhibitory activity was increased about 4-fold as reported. On the other hand, the ACE inhibitory activity of the casein-miso of the present invention was 948 U/g, which means that the ACE inhibitory activity of the casein-miso was not only increased 7.8-fold as compared with the normal miso but increased 1.9-fold as compared with the ACE inhibitory of the skimmed milk-miso.

TABLE 2
Comparison of the conditions for producing miso

| | normal | skimmed | casein- |
|---|---|---|---|
| | miso | milk-miso | miso |
| casein or | — | 16.0% | 16.0% |
| powdered skimmed milk | | | |
| NaCl concentration | 12.0% | 10.8% | 10.8% |
| water content | 43.0% | 45.2% | 45.2% |

| | | | | | | |
|---|---|---|---|---|---|---|
| ripening period | 60 | days | 60 | days | 60 | days |
| ripening temperature | 30° | C. | 30° | C. | 30° | C. |

TABLE 3
Comparison of ACE inhibitory activities among
normal miso, skimmed milk-miso and casein-miso

| | normal | skimmed | casein- |
|---|---|---|---|
| | miso | milk-miso | miso |
| IC50 (mg/ml) | 8.2 | 2.0 | 1.1 |
| ACE inhibitory activity | 122 | 511 | 948 |

To investigate the effects of casein concentration on the production of the peptides with ACE inhibitory activity in the resulting misos, the ACE inhibitory activities of the miso produced according to the formulation shown in Table 1 (II) in “[Methods of producing various miso]” section were evaluated (FIG. 2). As shown in FIG. 2, a miso with high ACE inhibitory activity was constantly obtained when the additive amount of casein was 10% (by weight) or more.

### Example 2

### Analysis of the Sizes of the Peptides Contained in the Miso of the Present Invention

To investigate the characteristics of the peptides with ACE inhibitory activity produced during the production of the casein-miso, the following experiments were conducted. Firstly, to estimate the molecular sizes of the contained peptides, the miso-peptides in the casein-miso obtained in Example 1 were subjected to scan-mode analysis of LC/MS (Shimazu, LCMS-2010A), to determine the distribution of detected peptides. The resolving condition was as follows:

### [Condition for LC]

Eluent A: Ultra-pure water+0.1% formic acid
Eluent B: acetonitrile+0.1% formic acid
gradient: 0-50 minutes (B: 0-100%)

### [Condition for MS]

Scan mode for [M+H]⁺
m/z Detection range: 50-2000

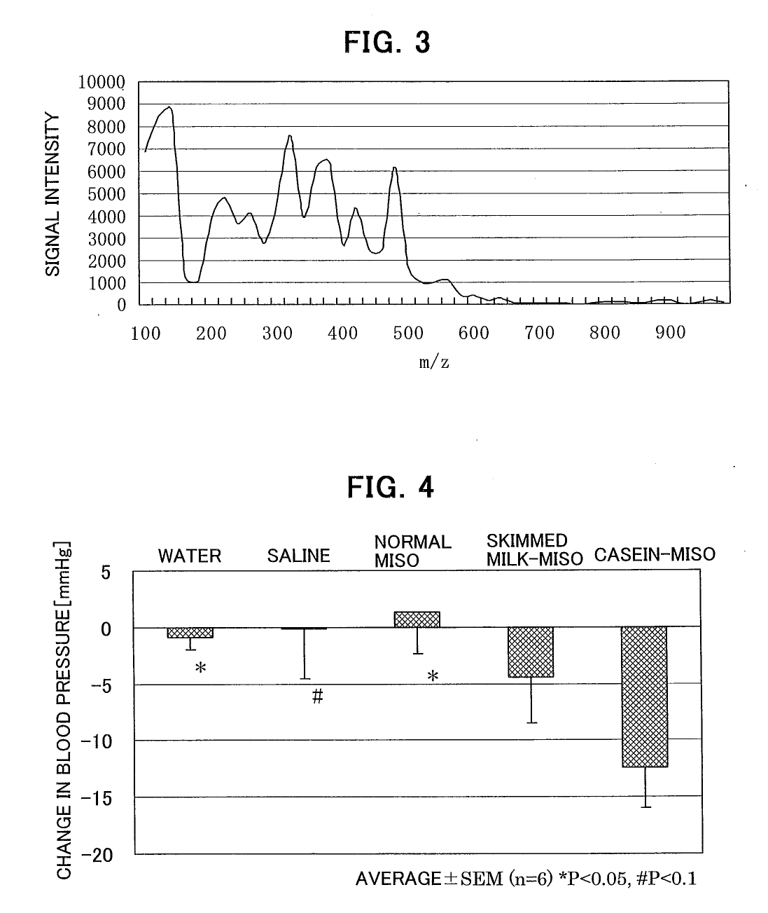

The analyzed results of the molecular sizes of the peptides by LS/MS scan mode were shown in FIG. 3. Since few signals with m/z 500 or more were observed, it was determined that the casein-miso mainly contained peptides having a molecular weigh of 500 or less. By calculating the area below the signal intensity curve it was shown that in the casein-miso peptides with a molecular weigh of 500 or less (corresponding to peptides with about 5 amino acid residues or less) were contained at a ratio of 90% or more, which means an ideal population of short peptides which exhibits ACE inhibitory activity.

### Example 3

### Analysis of Amino Acid Composition of the Peptides Contained in the Miso According to the Present Invention

Forty μl of the miso-peptides obtained in Example 1 were hydrolyzed in 200 μl of 6N HCl at 110° for 22 hours, dried under reduced pressure and the residue was dissolved in 100 μl of pure water and filtered through a 0.22 μm filter. The filtrate was diluted 10-fold and 50 μl thereof was analyzed by specialized amino acid analyzing system/ninhydrin coloring b using amino acid analyzer (Hitachi amino acid analyzer L-8500). As a result, it was revealed that the peptides in the casein-miso the content of Pro was as high as 16% or more, as shown in Table 4.

TABLE 4
Analysis of amino acids in casein-miso

| Amino acid | content in the casein-miso (%) |
|---|---|
| Asp (aspartic acid) | 9.4 |
| Thr (threonine) | 4.6 |
| Ser (serine) | 9.9 |
| Glu (glutamic acid) | 18.8 |
| Gly (glycine) | 5.5 |
| Ala (alanine) | 2.4 |
| ½Cys (cysteine) | 0.7 |
| Val (valine) | 6.4 |
| Met (methionine) | 1.0 |
| Ile (isoleucine) | 8.0 |
| Leu (leucine) | 4.3 |
| Tyr (tyrosine) | 2.2 |
| Phe (phenylalanine) | 4.7 |
| Lys (lysine) | 2.5 |
| His (histidine) | 1.6 |
| Trp (tryptophan) | less than 0.1 |
| Arg (arginine) | 1.8 |
| Pro (proline) | 16.2 |

It has been generally reported that peptides exhibiting ACE inhibitory activity are likely to be short peptides or such peptides enriched with Pro residue have been reported. Thus the miso-peptides were subjected to amino acid sequencing analysis as a mixture of peptides to analyze the composition of the peptides produced during the production of the casein-miso.

The casein-miso peptides obtained in Example 1 were diluted 50-fold and 5 μl thereof was analyzed from N-terminal to 10 cycles by using the following apparatus:

Protein Sequencer: Procise cLC 492cLC (Applied Biosystems)

PTH analyzer: 140D (Applied Biosystems)

Analysis Program Pulsed-liquid cLC

TABLE 5
Results of analysis of amino acid sequences

| | 1^{st} | 2^{nd} | 3^{rd} | 4^{th} | 5^{th} |
|---|---|---|---|---|---|
| Amino acid | residue | residue | residue | residue | residue |
| Ala (alanine) | 2.84 | 1.67 | 0.84 | 1.40 | 1.18 |
| Asp (aspartic acid) | 13.66 | 5.43 | 6.49 | 6.71 | 6.84 |
| Glu (glutamic acid) | 7.66 | 9.67 | 10.35 | 17.92 | 21.27 |
| Phe (phenylalanine) | 4.91 | 1.63 | 0.99 | 0.80 | 0.68 |
| Gly (glycine) | 6.12 | 13.36 | 14.95 | 16.12 | 12.77 |
| His (histidine) | 1.25 | 2.52 | 3.09 | 4.26 | 3.84 |
| Ile (isoleucine) | 13.03 | 12.70 | 13.69 | 7.64 | 5.88 |
| Lys (lysine) | 3.02 | 3.12 | 2.49 | 2.20 | 1.97 |
| Leu (leucine) | 2.90 | 4.32 | 2.21 | 1.58 | 1.33 |
| Met (methionine) | 0.72 | 0.64 | 0.83 | 0.58 | 0.47 |
| Asn (asparagine) | 5.95 | 2.82 | 1.94 | 2.46 | 2.26 |
| Pro (proline) | 1.97 | 18.36 | 15.82 | 13.88 | 9.65 |
| Gln (glutamine) | 0.88 | 3.96 | 3.15 | 3.81 | 4.44 |
| Arg (arginine) | 4.50 | 4.11 | 4.93 | 5.14 | 6.08 |
| Ser (serine) | 9.54 | 3.98 | 5.52 | 6.35 | 12.73 |
| Thr (threonine) | 2.51 | 3.62 | 3.78 | 3.98 | 4.44 |
| Val (valine) | 5.80 | 4.19 | 3.79 | 2.26 | 2.09 |
| Trp (tryptophan) | 4.28 | 1.02 | 0.42 | 0.21 | 0.00 |
| Tyr (tyrosine) | 8.28 | 2.89 | 4.70 | 2.72 | 2.07 |
| Total (%) | 100.00 | 100.00 | 100.00 | 100.00 | 100.00 |

The results of the analysis revealed that proline (Pro) was contained at the second and third residue at a ratio of 18% and 16%, respectively. That is, it can be said that the miso according to the present invention contained peptides having proline at the second and third amino acid residues from the N-terminal of all the peptides at a ratio of 17% on average. Additionally, it was supposed that the ACE inhibitory effect of the miso-peptides was high because the peptides contained small molecular peptides with 5 or less amino acid residues and also contained Pro within the sequences.

### Example 4

### Flavor Evaluation of Casein-Miso and Skimmed Milk-Miso

Sensory evaluation was conducted for casein-miso and skimmed milk-miso produced according to the formulation shown in Table 1 (I) by 17 persons. The sensory evaluation comparison was conducted by 2-point discrimination method using 17 g of misos dissolved in 180 ml of hot water.

Twelve persons felt that casein-miso was better in taste and flavor, 5 persons felt that skimmed milk-miso was better, and there was a tendency that the casein-miso was organoleptically preferable.

### <Preparation of Processed Miso>

Using the casein-miso produced according to the formulation shown in Table 1 (I) according to Example 1, a processed miso with additive seasonings was produced. According to the formulation shown in Table 6, the casein-miso, powdered dried bonito, bonito extract, kelp extract, spirit, amino acids base seasonings and adjusting water were mixed. Then the mixture was heated at 85° C. for 5 minutes to avoid degradation of the seasoning including the powdered dried bonito and the like by enzymes in the miso.

To 20 g of such instant miso with additive seasonings was dissolved in 160 ml of hot water and subjected to sensory evaluation. As a result, it was favorable in the taste and flavor and was preferable to eat. The instant miso prepared herein was confirmed to contain ACE inhibitory activity of 9,400 U or more in total per 20 g per serving.

TABLE 6
Formulation of the instant miso

| raw materials | content (%) |
|---|---|
| casein-miso | 72.0 |
| powdered dried bonito | 1.6 |
| bonito extract | 1.1 |
| kelp extract | 0.8 |
| spirit | 3.0 |
| amino acid base seasoning | 1.1 |
| adjusting water | 10.4 |

### Example 5

### Production of Freeze-Dried Foods

A freeze-dried food containing the processed miso prepared in Example 4 and various materials was produced according to the formulation shown in Table 7. Firstly, the materials were blanched and mixed depending on the experimental groups as shown in Table 7. Experimental group 1 is mainly formulated with green onion, group 2 was mainly formulated with bean curd (“tofu”) and group 3 was mainly formulated with eggplant. The indicated amount of water was then added to the processed miso of Example 4 to dissolve the miso.

The indicated amount of the blanched materials and the dissolved miso were admixed and pre-freezed at −30° C. for 10 hours or more. Then it was dried at 80-85° C. for 24 hours under the condition where the temperature of the product did not exceed 50° C.

Such produced freeze-dried food of each group was dissolve in 160 ml of hot water per serving to prepare a miso soup which was then subjected to sensory evaluation. The results revealed that no groups were organoleptically unfavorable and that the casein-miso of the present invention is organoleptically suitable for a wide range of materials.

TABLE 7
Formulation of the freeze-dried foods (formulation per serving)

| | use | water | Materials (g) | | | | |
|---|---|---|---|---|---|---|---|
| Experimental | processed | addition | green | egg- | | fried | brown |
| group | miso (g) | (g) | onion | plant | tofu | tofu | seaweed |
| 1 | 15.8 | 17.5 | 22.1 | — | — | 3.2 | 0.4 |
| 2 | 15.8 | 17.5 | 9.5 | — | 8.4 | — | |
| 3 | 15.8 | 17.5 | 11.0 | 11.5 | — | 0.8 | |

### Example 6

### Verification of Hypotensive Effect of the Casein-Miso

The casein-miso, normal miso and skimmed milk-miso produced according to the formulation of Table 1 (I) in Example 1 were evaluated for their hypotensive effect as described below. Male spontaneously hypertensive rats of 19-21 week old (SHR: Japan Charles River) were sufficiently acclimated under inverted day-night cycle 8:00 AM/PM condition before measuring blood pressure. For SHR those corresponding to systolic pressure of +/−5 mmHg on average as measured by blood pressure determination were selected to compose a group consisting of six rats. Blood pressure determination for SHRs was for evaluating decreased value in blood pressure 5 hours post-forced oral administration of various miso homogenates to SHR at a dose of 0.6 g/aminal (1.8 g/kg). The results confirmed a significant decrease in blood pressure for casein-miso as compared with the control group received water (significant difference: *P<0.05). On the other hand average blood pressure also decreased for skimmed milk-miso groups, but the decrease was not significant as compared with the control groups. Similarly no significant decrease in blood pressure was observed for the normal miso. These results were nearly proportional to the in vitro ACE inhibitory activities.

As shown by the results in the examples, the hypertensive effect of the casein-miso of the present invention could be confirmed both in vitro and in vivo. The tests herein is not intended to demonstrate a minimal effective amount the casein-miso according to the present invention on SHR and it is expected that less amount of ingestion of the casein-miso may be effective in human.

### REFERENCES

1. JP 06-4-0944
2. WO 2005/074712
3. Yamamoto et al. (2003) Current Pharmacol Des. 9: 1345-1355
4. Muguruma et al., Zoukyo, Vol. 100 (2005) pp. 216-223

1-19. (canceled)

20. A method of producing a miso, comprising adding animal milk at a ratio of 10% (by weight) or more during a mixing step, or fermentation and ripening step of the production of miso.

21. The method according to claim 20, wherein 90% or more of peptides of total peptides contained in the miso are peptides having a molecular weight of 500 or less.

22. The method according to claim 20, wherein Pro content is 15% or more based on amino acids composing total peptides contained in the miso.

23. The method according to claim 20, wherein Pro content in peptides contained in the miso is 15% as the N-terminal second and/or third amino acid residue of the peptides based on all amino acids.

24. The method according to claim 20, wherein the miso comprises 550 U/g or more angiotensin converting enzyme inhibitory activity.

25. A miso comprising a brew containing animal milk casein at a ratio of 10% (by weight) or more, said casein being added during a mixing step or a fermentation and ripening step.

26. The miso according to claim 25, wherein 90% or more peptides of total peptides contained in the brew are peptides having a molecular weight of 500 or less.

27. The miso according to claim 25, wherein Pro content is 15% or more based on amino acids composing total peptides contained in the miso.

28. The miso according to claim 25, wherein Pro content in peptides contained in the brew is 15% or more as the N-terminal second and/or third amino acid residue of the peptides based on all amino acids.

29. The method according to claim 20, wherein the brew comprises 550 U/g or more angiotensin converting enzyme inhibitory activity.

30. A method or producing a food, comprising adding a seasoning and/or a food to the miso according to claim 20, thereby the food is processed such that the food has 7,800 U or more of an angiotensin converting enzyme inhibitory activity per serving.

31. A method of producing a food for decreasing blood pressure or controlling an increase of blood pressure.

32. The miso according to claim 25, further comprising the step of powderization of the miso.

33. The miso according to claim 32, wherein the powderization is conducted by freeze-drying.

34. A food comprising the miso according to claim 25 and having 7,800 U or more of an angiotensin converting enzyme inhibitory activity per serving.

35. A food according to claim 34, wherein the miso is powdered.
